SEQUENCE LISTING (92 sequences)

SEQ ID NO: 1 - protein, 213 aa, Mus musculus
QIVLTQSPAIMSASPGEKVTMTCSASSSVSYMNWYQQKSGTSPKRWIYDTSKLASGVPAHFRGSGSGTSYSLTISGMEAEDAATYYCQQWSSNPFTFGSGTKLEINRADTAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 2 - protein, 449 aa, Mus musculus
QVQLQQSGAELARPGASVKMSCKASGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSSAKTTAPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIEPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 3 - protein, 15 aa, Mus musculus
SGTKLEINRADTAPT

SEQ ID NO: 4 - protein, 16 aa, Mus musculus
GSGTKLEINRADTAPT

SEQ ID NO: 5 - protein, 13 aa, Mus musculus
VKWKIDGSERQNG

SEQ ID NO: 6 - protein, 14 aa, Mus musculus
NVKWKIDGSERQNG

SEQ ID NO: 7 - protein, 15 aa, Mus musculus
INVKWKIDGSERQNG

SEQ ID NO: 8 - protein, 16 aa, Mus musculus
INVKWKIDGSERQNGV

SEQ ID NO: 9 - protein, 17 aa, Mus musculus
WPSQSITCNVAHPASST

SEQ ID NO: 10 - protein, 15 aa, Mus musculus
KDSTYSMSSTLTLTK

SEQ ID NO: 11 - protein, 16 aa, Mus musculus
KDSTYSMSSTLTLTKD

SEQ ID NO: 12 - protein, 17 aa, Mus musculus
KDSTYSMSSTLTLTKDE

SEQ ID NO: 13 - protein, 17 aa, Mus musculus
SKDSTYSMSSTLTLTKD

SEQ ID NO: 14 - protein, 18 aa, Mus musculus
SKDSTYSMSSTLTLTKDE

SEQ ID NO: 15 - protein, 18 aa, Mus musculus
DSKDSTYSMSSTLTLTKD

SEQ ID NO: 16 - protein, 19 aa, Mus musculus
DSKDSTYSMSSTLTLTKDE

SEQ ID NO: 17 - protein, 20 aa, Mus musculus
DQDSKDSTYSMSSTLTLTKD

SEQ ID NO: 18 - protein, 21 aa, Mus musculus
DQDSKDSTYSMSSTLTLTKDE

SEQ ID NO: 19 - protein, 22 aa, Mus musculus
TDQDSKDSTYSMSSTLTLTKDE

SEQ ID NO: 20 - protein, 15 aa, Mus musculus
SGTKLEINRADTAPT

SEQ ID NO: 21 - protein, 16 aa, Mus musculus
GSGTKLEINRADTAPT

SEQ ID NO: 22 - protein, 14 aa, Mus musculus
WPSQSITCNVAHPA

SEQ ID NO: 23 - protein, 15 aa, Mus musculus
WPSQSITCNVAHPAS

SEQ ID NO: 24 - protein, 17 aa, Mus musculus
WPSQSITCNVAHPASST

SEQ ID NO: 25 - protein, 18 aa, Mus musculus
WPSQSITCNVAHPASSTK

SEQ ID NO: 26 - protein, 17 aa, Mus musculus
INVKWKIDGSERQNGVL

SEQ ID NO: 27 - protein, 13 aa, Homo sapiens
QFQKEDAALTIYE

SEQ ID NO: 28 - protein, 16 aa, Homo sapiens
QLQQFQKEDAALTIYE

SEQ ID NO: 29 - protein, 17 aa, Homo sapiens
KQLQQFQKEDAALTIYE

SEQ ID NO: 30 - protein, 11 aa, Homo sapiens
NVYHQINHLKT

SEQ ID NO: 31 - protein, 12 aa, Homo sapiens
NVYHQINHLKTV

SEQ ID NO: 32 - protein, 13 aa, Homo sapiens
NVYHQINHLKTVL

SEQ ID NO: 33 - protein, 14 aa, Homo sapiens
NVYHQINHLKTVLE

SEQ ID NO: 34 - protein, 15 aa, Homo sapiens
NVYHQINHLKTVLEE

SEQ ID NO: 35 - protein, 16 aa, Homo sapiens
NVYHQINHLKTVLEEK

SEQ ID NO: 36 - protein, 12 aa, Homo sapiens
ANVYHQINHLKT

SEQ ID NO: 37 - protein, 13 aa, Homo sapiens
ANVYHQINHLKTV

SEQ ID NO: 38 - protein, 14 aa, Homo sapiens
ANVYHQINHLKTVL

SEQ ID NO: 39 - protein, 15 aa, Homo sapiens
ANVYHQINHLKTVLE

SEQ ID NO: 40 - protein, 16 aa, Homo sapiens
ANVYHQINHLKTVLEE

SEQ ID NO: 41 - protein, 17 aa, Homo sapiens
ANVYHQINHLKTVLEEK

SEQ ID NO: 42 - protein, 14 aa, Homo sapiens
LANVYHQINHLKTV

SEQ ID NO: 43 - protein, 15 aa, Homo sapiens
LANVYHQINHLKTVL

SEQ ID NO: 44 - protein, 16 aa, Homo sapiens
LANVYHQINHLKTVLE

SEQ ID NO: 45 - protein, 17 aa, Homo sapiens
LANVYHQINHLKTVLEE

SEQ ID NO: 46 - protein, 16 aa, Homo sapiens
LLANVYHQINHLKTVL

SEQ ID NO: 47 - protein, 17 aa, Homo sapiens
LLANVYHQINHLKTVLE

SEQ ID NO: 48 - protein, 18 aa, Homo sapiens
LLANVYHQINHLKTVLEE

SEQ ID NO: 49 - protein, 19 aa, Homo sapiens
LLANVYHQINHLKTVLEEK

SEQ ID NO: 50 - protein, 18 aa, Homo sapiens
NLLANVYHQINHLKTVLE

SEQ ID NO: 51 - protein, 19 aa, Homo sapiens
NLLANVYHQINHLKTVLEE

SEQ ID NO: 52 - protein, 13 aa, Homo sapiens
ILRNFYFINRLTG

SEQ ID NO: 53 - protein, 15 aa, Homo sapiens
VEILRNFYFINRLTG

SEQ ID NO: 54 - protein, 11 aa, Homo sapiens
NVYHQINHLKT

SEQ ID NO: 55 - protein, 12 aa, Homo sapiens
NVYHQINHLKTV

SEQ ID NO: 56 - protein, 13 aa, Homo sapiens
NVYHQINHLKTVL

SEQ ID NO: 57 - protein, 14 aa, Homo sapiens
NVYHQINHLKTVLE

SEQ ID NO: 58 - protein, 15 aa, Homo sapiens
NVYHQINHLKTVLEE

SEQ ID NO: 59 - protein, 12 aa, Homo sapiens
ANVYHQINHLKT

SEQ ID NO: 60 - protein, 13 aa, Homo sapiens
ANVYHQINHLKTV

SEQ ID NO: 61 - protein, 14 aa, Homo sapiens
ANVYHQINHLKTVL

SEQ ID NO: 62 - protein, 15 aa, Homo sapiens
ANVYHQINHLKTVLE

SEQ ID NO: 63 - protein, 13 aa, Homo sapiens
LANVYHQINHLKT

SEQ ID NO: 64 - protein, 14 aa, Homo sapiens
LANVYHQINHLKTV

SEQ ID NO: 65 - protein, 15 aa, Homo sapiens
LANVYHQINHLKTVL

SEQ ID NO: 66 - protein, 16 aa, Homo sapiens
LANVYHQINHLKTVLE

SEQ ID NO: 67 - protein, 14 aa, Homo sapiens
LLANVYHQINHLKT

SEQ ID NO: 68 - protein, 17 aa, Homo sapiens
LLANVYHQINHLKTVLE

SEQ ID NO: 69 - protein, 15 aa, Homo sapiens
NLLANVYHQINHLKT

SEQ ID NO: 70 - protein, 18 aa, Homo sapiens
NLLANVYHQINHLKTVLE

SEQ ID NO: 71 - protein, 11 aa, Homo sapiens
RNFYFINRLTG

SEQ ID NO: 72 - protein, 13 aa, Homo sapiens
ILRNFYFINRLTG

SEQ ID NO: 73 - protein, 14 aa, Homo sapiens
ILRNFYFINRLTGY

SEQ ID NO: 74 - protein, 14 aa, Homo sapiens
EILRNFYFINRLTG

SEQ ID NO: 75 - protein, 15 aa, Homo sapiens
VEILRNFYFINRLTG

SEQ ID NO: 76 - protein, 15 aa, Homo sapiens
LQQFQKEDAALTIYE

SEQ ID NO: 77 - protein, 16 aa, Homo sapiens
QLQQFQKEDAALTIYE

SEQ ID NO: 78 - protein, 17 aa, Homo sapiens
KQLQQFQKEDAALTIYE

SEQ ID NO: 79 - protein, 18 aa, Homo sapiens
IKQLQQFQKEDAALTIYE

SEQ ID NO: 80 - protein, 19 aa, Homo sapiens
IKQLQQFQKEDAALTIYEM

SEQ ID NO: 81 - protein, 19 aa, Homo sapiens
EIKQLQQFQKEDAALTIYE

SEQ ID NO: 82 - protein, 22 aa, Homo sapiens
IPEEIKQLQQFQKEDAALTIYE

SEQ ID NO: 83 - protein, 13 aa, Homo sapiens
ILRNFYFINRLTG

SEQ ID NO: 84 - protein, 15 aa, Homo sapiens
VEILRNFYFINRLTG

SEQ ID NO: 85 - protein, 13 aa, Homo sapiens
ILRNFYFINRLTG

SEQ ID NO: 86 - protein, 14 aa, Homo sapiens
EILRNFYFINRLTG

SEQ ID NO: 87 - protein, 15 aa, Homo sapiens
VEILRNFYFINRLTG

SEQ ID NO: 88 - protein, 16 aa, Mus musculus
WPSQSITCNVAHPASS

SEQ ID NO: 89 - protein, 10 aa, Artificial Sequence
  Description of Artificial Sequence Synthetic peptide
KLKLKLKLKL

SEQ ID NO: 90 - protein, 9 aa, Artificial Sequence
  Description of Artificial Sequence Synthetic peptide
MNMNMNMNM

SEQ ID NO: 91 - protein, 11 aa, Artificial Sequence
  Description of Artificial Sequence Synthetic peptide
PQPQPQPQPQP

SEQ ID NO: 92 - protein, 8 aa, Artificial Sequence
  Description of Artificial Sequence Synthetic peptide
MNMNMNMN

(19) United States
(12) Patent Application Publication
(10) Pub. No.: US 2006/0251664 A1
(43) Pub. Date: Nov. 9, 2006

(54) Method for the identification of epitopes related to immunogenicity in biopharmaceuticals

(76) Inventors: Harald Kropshofer, Loerrach (DE); Anne Vogt, Loerrach (DE)

Correspondence Address: HOFFMANN-LA ROCHE INC.;PATENT LAW DEPARTMENT, 340 KINGSLAND STREET, NUTLEY, NJ, 07110, US

(21) Appl. No.: 11/404,253

(22) Filed: Apr. 13, 2006

(30) Foreign Application Priority Data
Apr. 20, 2005 (EP) 05103199.5

(51) Int. Cl. G01N 33/567; A61K 39/00

(52) U.S. Cl. 424/184.1; 435/7.2

(57) ABSTRACT

The present invention relates to a method for identifying peptides involved in immunogenicity comprising the steps of a) providing cells expressing antigen presenting receptors (APR) in a number providing 0.1 to 5 ug of APR molecules, b) contacting the cells from (a) with a source of immunogenic peptides, c) isolating APR molecule-immunogenic pep tide complexes from the cells, d) eluting the associated peptides from the APR molecules, e) identifying the immunogenic peptides, and f) verifying the identified immunogenic peptides as epitopes.

### PRIORITY TO RELATED APPLICATIONS

This application claims the benefit of European Application No. 05103199.5, filed Apr. 20, 2005, which is hereby incorporated by reference in its entirety.

### BACKGROUND OF THE INVENTION

One aspect that contributes to the immunotoxicity of biological therapeutics is their immunogenicity. Biopharmaceuticals that are immunogenic give rise to antibodies that may lead to potency loss and adverse events, such as allergy, infusion reactions or autoimmunity, in clinical trials. The potential to be immunogenic relies on the presence of T cell epitopes within the sequence of a protein pharmaceutical. Current methods used so far rely on in silico prediction algorithms, in vitro screening of overlapping synthetic peptides in T cell activation assays or animal vaccination models.

Activation of CD4+ T cells is only accomplished when T cell epitopes are presented in the context of molecules encoded by the major histocompatibility complex (MHC). In humans, MHC molecules are termed human leukocyte antigens (HLA). HLA-associated peptides are short, encompassing 9-25 amino acids (Kropshofer, H. & Vogt, A. B., Immunol Today 18 (1997) 77-82).

With regard to their function, two classes of MHC-peptide complexes can be distinguished (Germain, R., Cell 76 (1994) 287-299): (i) MHC class I-peptide complexes can be expressed by almost all nucleated cells in order to attract CD8+ cytotoxic T cells which lyse infected cells or tumor cells, (ii) MHC class II-peptide complexes are constitutively expressed only on so-called antigen presenting cells (APCs), such as B lymphocytes, macrophages or dendritic cells (DCs). In particular, DCs have the capacity to prime CD4+ T helper cells and thereby initiate immunogenicity (Banchereau, J. & Steinman, R. M., Nature 392 (1998) 245-254).

However, the amount of MHC molecules/complexes necessary for such methods (re: screening of synthetic peptides or T cell activation assays) is at least about 200 ug MHC class II molecules derived from an unlimited source (inbred mice) (Dongre A R et al., EJI 2001, 31, 1485-94). This is about two orders of magnitude more material than available from human peripheral blood.

### SUMMARY OF THE INVENTION

The present invention provides a method for isolating and identifying peptides that may render biopharmaceuticals immunogenic after administration to humans. The method provides complexes of peptide receptors with potentially immunogenic peptides in an amount of 0.1 to 5 μg, preferably in an amount of 0.2 to 3 μg. This quantity is approximately equal to the amount of material which is normally available from DCs cells obtained from peripheral blood of patients or healthy donors.

Specifically, the present invention provides a method for identifying peptides involved in immunogenicity comprising the steps of
a) providing cells expressing antigen presenting receptors (APR) in a number providing 0.1 to 5 ug receptors (molecules), preferably, in a number providing 0.2 to 3 ug,
b) contacting the cells from (a) with a source of immunogenic peptides,
c) isolating APR-immunogenic peptide complexes from the cells,
d) eluting the associated immunogenic peptides from the APR,
e) identifying the immunogenic peptides, and optionally,
f) validating the identified immunogenic peptides as epitopes.

Preferably, the method for identifying peptides involved in immunogenicity comprises the steps of
a) providing cells expressing antigen presenting receptors (APR) in a number providing 0.1 to 5 ug receptors (molecules), preferably, in a number providing 0.2 to 3 ug,
b) contacting the cells from (a) with a source of immunogenic peptides,
c) sequestering the APR-immunogenic peptide complexes from the cells by immunoprecipitation or immunoaffinity chromatography,
d) washing the bounded complexes of APR with antigenic peptides with water or low salt buffer,
e) eluting the associated peptides from the APR,
f) identifying the immunogenic peptides, and optionally,
g) validating the identified immunogenic peptides as epitopes.

Preferably, the antigen presenting receptor is a MHC II molecule.

### DESCRIPTION OF THE FIGURES

FIG. 1 shows a diagram of the methodology to study naturally processed MHC class II-associated peptide epitopes derived from a therapeutic polypeptide added to human dendritic cells.

FIG. 2 show a comparison of OKT3-derived peptide epitopes identified through the in silico prediction algorithm TEPITOPE versus the in vitro methodology involving dendritic cells. Potential T cell epitopes were predicted for the HLA-DRB1 alleles *0301, *0401, *0701 and *1101, as indicated by small black rectangles above the protein sequence. The threshold for the TEPITOPE analysis was set to 1-4%. The signal peptide of unprocessed OKT3 light chain was omitted. The epitopes identified by the cellular in vitro technology are marked by numbers and boxes in the OKT3 sequence.

FIG. 3 shows a diagram of CD4+ T cell activation by synthetic OKT3-derived peptides #1-4. The intensity of T cell activation is indicated by the stimulation index (SI). The sequences of the peptides used for stimulation (10 uM each) were as follows: #1, OKT3-lc 98-113, GSGTKLEINRADTAPT, #2, OKT-lc 143-158, INVKWKIDGSERQNGV, #3, OKT3-hc 194-209, WPSQSITCNVAHPASS, #4, OKT3-lc 164-183, DQDSKDSTYSMSSTLTLTKDE. T cells were re-stimulated once (B) or twice (A,C) with the respective peptide and mature dendritic cells. The HLA-DRB1 genotypes of the dendritic cells and T cells employed are indicated on top of each diagram. Error bars indicate SD obtained with 3 independent experiments. The average SI in the absence of added peptide was adjusted to 1.0. Donor cells with the following DRB1 haplotype were used: *0401/*0701 (A), *0301/*1501 (B), and *1001/*1201 (C)

### DETAILED DESCRIPTION OF THE INVENTION

This invention relates to an in vitro method for identifying epitopes that may play a causal role in inducing immunogenicity of biopharmaceuticals, such as antibodies or other therapeutic proteins. More specifically, the method of the invention can be used for determining the sequence of immunogenic peptides presented via peptide receptors of dendritic cells which trigger immune reactions leading to immunogenicity. Knowledge about immunogenic epitopes opens the possibility to de-risk therapeutic polypeptides by site-directed mutagenesis with the aim to generate non-immunogenic biopharmaceuticals.

The present invention relates to methods useful for determining epitopes that may render pharmaceutical proteins immunogenic, based on isolating immunogenic peptides from human dendritic cells that have been pulsed with the respective pharmaceutical protein, and the determination of the sequence of the potential T cell epitopes of the pharmaceutical protein. The method of the present invention can be utilized for identification of immunogenic epitopes contained in engineered polypeptides, antibodies or other therapeutic proteins.

Almost any therapeutic protein displays a certain degree of immunogenicity in clinical trials. The initial trigger for immunogenicity is the activation of CD4+ T lymphocytes upon recognition of peptide fragments of the respective pharmaceutical protein. These peptides are referred to as “T cell epitopes” or, briefly, “epitopes”.

Activation of CD4+ T cells is only accomplished when T cell epitopes are presented in the context of molecules encoded by the major histocompatibility complex (MHC). In humans, MHC molecules are termed human leukocyte antigens (HLA). HLA-associated peptides are short, encompassing 9-25 amino acids (Kropshofer, H. & Vogt, A. B., Immunol Today 18 (1997) 77-82).

With regard to their function, two classes of MHC-peptide complexes can be distinguished (Germain, R., Cell 76 (1994) 287-299): (i) MHC class I-peptide complexes can be expressed by almost all nucleated cells in order to attract CD8+cytotoxic T cells which lyse infected cells or tumor cells, (ii) MHC class II-peptide complexes are constitutively expressed only on so-called antigen presenting cells (APCs), such as B lymphocytes, macrophages or dendritic cells (DCs). In particular, DCs have the capacity to prime CD4+ T helper cells and thereby initiate immunogenicity (Banchereau, J. & Steinman, R. M., Nature 392 (1998) 245-254).

Hence, the present innovative approach to identify immunogenicity hot spots in pharmaceutical proteins is to use DCs pulsed with the biopharmaceutical of choice and determine the sequence of the peptides associated to MHC class II molecules on DCs. In order to determine the potential immunogenicity of a biopharmaceutical in a manner that applies to the whole population, DCs from a series of blood donors have to be used that are representative for the variety in MHC class II genotypes of the respective population.

The present invention thus provides methods for isolating and identifying femtomolar amounts of potentially immunogenic peptide antigens derived from pharmaceutical proteins. Said method concerns immunogenicity monitoring of therapeutic proteins, e.g. polypeptides, monoclonal antibodies or other proteins. The method of the invention has the advantage that the identity of bound and/or presented peptides can be elucidated from the small quantity of dendritic cells that can be obtained from usual amounts of peripheral blood of a healthy donor. The described method ensures that the immunogenic peptides isolated and identified are those that are naturally-processed and presented by DCs in vitro upon encounter of a therapeutic protein.

### DEFINITIONS

The term “Antigen presenting receptors” or “APR” as used herein refers to a peptide receptor which binds antigenic peptides and presents them to other immunological cells and thereby mediating a specific humoral immune response. Preferred antigen presenting receptors are MHC class II molecules. MHC class II molecules include but are not limited to HLA-DR, HLA-DQ and HLA-DP molecules. Alternative APR that may play a role are the receptors of the CD1 family or other so far undefined receptors that present potentially immunogenic peptides to CD4+ helper T cells. Generally speaking, APR are surface molecules on APCs (antigen presenting cells, such as dendritic cells or B cells) which carry and present antigenic peptides (derived from antigenic proteins) to T lymphocytes. It is the MHC molecules, or HLA (human leucocyte antrigen) molecules in humans. They are composed of 2 subunits and may carry an antigenic peptide in the antigen binding site.

“APR molecule” is the same as “APR”, and “APR molecules” is used interchangeably with “APR”.

The term “polypeptide” as used herein refers to a chain of linked amino acids.

The term “immunogen” as used herein refers to any polypeptide that provokes an immune response when introduced into the body

The term “immunogenicity” as used herein refers to the quality of a substance which is able to provoke an immune response against the substance. A measure of how able the substance is at provoking an immune response against it.

The term “immunogenicity potential” as used herein refers to potential capacity of a polypeptide to elicit an immune response.

The term “immune response” as used herein refers to a bodily defense reaction that recognizes an invading substance and produces antibodies specific against that antigen.

All references cited herein are hereby referenced in their entirety.

### Detailed Description

The present invention provides a method for isolating and identifying peptides that may render biopharmaceuticals immunogenic after administration to humans. The method provides complexes of peptide receptors with potentially immunogenic peptides in an amount of 0.1 to 5 μg, preferably in an amount of 0.2 to 3 μg. This quantity equals to the amount of material which is normally available from DCs cells obtained from peripheral blood of patients or healthy donors. The lowest amount of material necessary in the prior art is about 200 μg MHC class II molecules derived from an unlimited source (inbred mice) (Dongre A R et al., EJI 2001, 31, 1485-94). This is about two orders of magnitude more material than available from human peripheral blood.

Specifically, the present invention provides a method for identifying peptides involved in immunogenicity comprising the steps of
a) providing cells expressing antigen presenting receptors (APR) in a number providing 0.1 to 5 ug receptors (molecules), preferably, in a number providing 0.2 to 3 ug,
b) contacting the cells from (a) with a source of immunogenic peptides,
c) isolating APR-immunogenic peptide complexes from the cells,
d) eluting the associated immunogenic peptides from the APR,
e) identifying the immunogenic peptides, and optionally,
f) validating the identified immunogenic pep tides as epitopes.

Preferably, the method for identifying peptides involved in immunogenicity comprises the steps of
a) providing cells expressing antigen presenting receptors (APR) in a number providing 0.1 to 5 ug receptors (molecules), preferably, in a number providing 0.2 to 3 ug,
b) contacting the cells from (a) with a source of immunogenic peptides,
c) sequestering the APR-immunogenic peptide complexes from the cells by immunoprecipitation or immunoaffinity chromatography,
d) washing the bounded complexes of APR with antigenic peptides with water or low salt buffer,
e) eluting the associated peptides from the APR,
f) identifying the immunogenic peptides, and optionally
g) validating the identified immunogenic peptides as epitopes.

Preferably, the antigen presenting receptor is a MHC II molecule.

Furthermore, the invention provides a method for decreasing the immunogenicity of a polypeptide comprising
a) identifying the immunogenic peptides of the polypeptide as described above
b) modifying the corresponding epitopes of the polypeptide so that the binding to APR molecules is reduced or abolished
c) thereby creating mutated polypeptide with reduced or no immunogenicity potential.

The modification of the corresponding epitope is achieved by exchanging of one or more amino acids. Preferably, these one or more amino acids are those responsible for anchoring the epitope to the APR. (see H. Kropshofer et al. EMBO J. 15, 6144-6154, 1996)

The amount of tissue or bodily fluid necessary to obtain e.g. 100 ng (or 0.1 μg) MHC class II molecules depends on the number of cells that do express MHC class II and on the expression rate of MHC class II molecules: e.g. 100 ng of MHC class II are equivalent to about 2×10⁵ mature DCs or about 5 to 10×10⁶peripheral blood monocytes or about 5×10⁷ peripheral blood mononuclear cells which can be obtained from about 50 ml of blood.

The high sensitivity required for identifying MHC class II associated peptides is explained by the fact that each type of these peptide receptors, e.g. human MHC class II gene product HLA-DR1, carries about 500 to 1000 different antigenic peptides (Chicz R M et al., J Exp. Med. 1993, 178, 27-47; Chicz R M & Urban R G, Immunol. Today, 1993, 15: 155-160). However, most of the 500 to 1000 different peptides attain very low copy numbers and, therefore, are not very likely to play a physiological role. Especially in the MHC class II field, those peptides that are of immunological relevance e.g. those that activate helper T cells and thereby facilitate immunogenicity of pharmaceutical proteins, attain moderate to high copy numbers (Latek R R & Unanue E R, Immunol. Rev. 1999, 172: 209-228). These peptides cover about 40 to 50% of the total amount of peptide material eluted from MHC class II molecules and equal to about 10 to 20 individual peptides.

Many MHC class II associated peptides are represented as a set of 2 to 5 C— and N-terminal truncation variants (Rudensky A Y et al, Nature 1992, 359, 429-431; Chicz et al. Nature 1992, 358: 764-768) sharing a common core sequence of about 10 to 13 amino acids which is essential for recognition by the T cell receptor. These truncation/elongation variants constitute the same T cell epitope. This means that the number of different epitopes, which are of importance is actually smaller, ranging from about 5 to 70 different epitopes. Thus, the abundance of immunogenic epitopes ranges from 0.2% to 5%.

Origin of the Peptides

The antigenic peptides of the present invention are peptides which are associated with MHC class II molecules on the surface of human DCs. The antigenic peptides may be bound to intra- or extracellular MHC class II molecules. The term “immunogenic peptide” as used herein refers to an antigenic peptide which may elicit an immune response. The immunogenic peptides may derive from polypeptides after coincubation with DCs. The polypeptides which are a potential source of immunogenic peptides are polypeptides including therapeutic polypeptides such as cytokines (i.e. interferones, interleukins, erythropoietin (Epo), granulocyte/macrophage colony-stimulating factor (GM-CSF) or tumor necrosis factor (TNF)), chemokines, growth factors, antibodies (i.e. monoclonal, polyclonal, chimeric and humanized antibodies), enzymes, structural elements, hormones and fragments thereof.

As all these peptide receptors are able to accommodate a broad variety of peptide ligands (see above), each single peptide whose sequence has to be determined is represented in only femtomolar amounts. 1 μg MHC class II (16 pmol) may carry dominant peptide species, with each single peptide attaining an occupancy of 0.1-2%, which equals to about 16-320 femtomoles. The methods of the present invention allow the isolation of these femtomolar amounts of potentially immunogenic peptides from 0.1 to 5 μg of antigen presenting receptors loaded with peptides and their subsequent sequencing.

Origin of the Antigen Presenting Receptors

The term “Antigen presenting receptors” or “APR” as used herein refers to a peptide receptor which binds antigenic peptides and presents them to other immunological cells and thereby mediating a specific humoral immune response. Preferred antigen presenting receptors are MHC class II molecules. MHC class II molecules include but are not limited to HLA-DR, HLA-DQ and HLA-DP molecules. Alternative APR that may play a role are the receptors of the CD1 family or other so far undefined receptors that present potentially immunogenic peptides to CD4+ helper T cells.

Origin of the Cellular Material

The methods of the present invention encompass all cells that express Antigen presenting receptors and at the same time are able to prime or activate CD4+ T cells. These cells are also referred to as antigen presenting cells (APCs) (Unanue, E. R. Macrophages, antigen presenting cells and the phenomena of antigen handling and presentation. In: Fundamental Immunology, 2nd edition (editor Paul, W. E) New York, Raven Press, 1989). Examples of APCs within the scope of the present invention comprise human B cells, human macrophages, and preferentially human dendritic cells. Additionally, cell mixtures that contain APC, such as peripheral blood mononuclear cells (PBMC) or peripheral blood lymphocytes (PBL) may also be used. Preferred APCs are cells expressing MHC class II molecules. Even more preferred APC are dendritic cells.

In order to judge the immunogenicity of polypeptides with respect to a certain population, a series of HLA-typed dendritic cells are preferentially to be used, with the HLA types representing the HLA frequencies of the whole population. For example, to cover the Caucasian population with regard to the HLA polymorphism at the HLA-DR locus, dendritic cells derived from about 15-20 blood donors differing in their HLA-DR genotype would be analyzed for potentially immunogenic peptides.

Solubilization of Antigen Presenting Receptors from Cells

For the purification of antigen presenting receptor-peptide complexes from cells, the membranes of the cells have to be solubilized. Cell lysis may be carried out with methods known in the art, e.g. freeze-and-thaw cycles and the use of detergents, and combinations thereof. Preferred lysis methods are solubilization using detergents, preferably TX-100, NP40, n-octylglucoside, Zwittergent, Lubrol, CHAPS, most preferably TX-100 or Zwittergent 3-12. Cell debris and nuclei have to be removed from cell lysates containing the solubilized receptor-peptide complexes by centrifugation. Therefore, in a further embodiment of the present invention, the complexes of antigen presenting receptors with immunogenic peptides are isolated from the cells with methods comprising solubilization with a detergent.

Nano-Scale Purification of MHC-Peptide Complexes

Furthermore, the invention provides the purification of the MHC-peptide complexes from cell lysates by methods comprising immunoprecipitation or immunoaffinity chromatography. For the immunoprecipitation or immunoaffinity chromatography, antibodies specific for MHC class II molecules and suitable for these methods are used. The specific antibodies are preferably monoclonal antibodies, and are covalently or non-covalently e.g. via Protein A, coupled to beads, e.g. sepharose or agarose beads. Examples of anti-HLA antibodies comprise:

anti-HLA-DR antibodies: L243, TU36, DA6.147, preferably L243; anti-HLA-DQ antibodies: SPVL3, TU22, TU169, preferably TU22 and TU169; anti-HLA-DP antibody B7/21, among others known to one of ordinary skill in the art.

Monoclonal antibodies specific for different MHC class II molecules may be commercially obtained (e.g. Pharmingen, Dianova) or purified from the supernatant of the respective hybridoma cells using Protein A- or Protein G-affinity chromatography. Purified monoclonal antibodies may be coupled by various methods known in the art, preferably by covalently coupling antibody amino groups to CNBr-activated sepharose.

Immunoisolation of MHC molecules may be performed by incubating the antibody-beads with the cell lysate under rotation for several hours or chromatographically by pumping the cell lysate through a micro-column. Washing of the antibody-beads may be performed in eppendorf tubes or in the microcolumn. The efficacy of the immunoprecipitation may be analyzed by SDS-PAGE and western blotting using antibodies recognizing denatured MHC molecules (anti-HLA-DRalpha: 1B5).

Elution and Fractionation of Antigen Presenting Receptor-Associated Peptides

By eluting the peptides from the receptor molecules, a complex mixture of naturally processed peptides derived from the source of potential immunogen and from polypeptides of intra- or extracellular origin, is obtained. Only after elution, peptides can be fractionated and subjected to sequence analysis.

The immunogenic peptides in the methods of the present invention may be eluted by a variety of methods known to one of ordinary skill in the art, preferably by using diluted acid, e.g., diluted acetonitrile (Jardetzky T S et al., Nature 1991 353, 326-329), diluted acetic acid and heating (Rudensky A Y et al., Nature 1991, 353, 622-626; Chicz R M et al., Nature 1992, 358, 764-768) or diluted trifluoro acetic acid at about 37° C. (Kropshofer H et al., J Exp Med 1992,175, 1799-1803). Most preferably, the peptides are eluted at 37° C. with diluted trifluoro acetic acid.

In a further embodiment, the sequestered antigen presenting receptor-peptide complexes are washed with water or low salt buffer before elution in order to remove residual detergent contaminants. The low salt buffer may be a Tris, phosphate or acetate buffer in a concentration range of 0.5-10 mM, preferably in a concentration of 0.5 mM. In a more preferred embodiment, the antigen presenting receptor-peptide complexes are washed with ultrapure water (sequencing grade) conventionally used for HPLC analysis, preferably with ultrapure (sequencing grade) water from MERCK. The washing step may be carried out by ultrafiltration. The ultrafiltration may be carried out in an ultrafiltration tube with a cut-off of 30 kD, 20 kD, 10 kD or 5 kD, preferably of 30 kD and a tube volume of 0.5-1.0 ml (“Ultrafree” tubes; Millipore). The washing in the ultrafiltration tube may be carried out 4 to 12 times, preferably 6 to 10 times, with a volume of 10 to 20 times the volume of the beads carrying the receptor-peptide complexes, preferably with a volume of 15 times the beads. The eluted peptides may be separated from the remaining antigen presenting receptor molecules using the same ultrafiltration tube. The eluted peptides may then be lyophilized.

Peptide Sequence Analysis by Liquid Chromatography-Mass Spectrometry (LC-MS)

In a further embodiment of the present invention, the isolated immunogenic peptides are fractionated, sequenced and identified. By sequencing it is understood that the amino acid sequence of the individual peptides in the mixture of isolated immunogenic peptides is elucidated by methods adequate to sequence femtomolar amounts of peptides. By identifying it is understood that it is established from which proteins or polypeptides the immunogenic peptides are derived and which sequence they constitute within these proteins or polypeptides.

In a first step, the complex mixture of eluted peptides may be fractionated by one of a variety of possible chromatographic methods, e.g. by reversed phase, anion exchange, cation exchange chromatography or a combination thereof. Preferably, the separation is performed by C18-reverse phase chromatography or by reversed-phase/cation exchange two-dimensional HPLC, denoted as MudPit (Washburn M P et al., Nat Biotechnol., (2001), 19,242-247).

The fractionation may be done in a HPLC mode utilizing fused-silica micro-capillary columns which are either connected to a nano-flow electrospray source of a mass spectrometer or to a micro-fractionation device which spots the fractions onto a plate for MALDI analysis.

A variety of mass spectrometric techniques are suitable, preferably MALDI-post source decay (PSD) MS or electrospray ionization tandem mass spectrometry (ESI-MS), most preferably ion-trap ESI-MS.

The sequences of the individual peptides can be determined by means known to one of ordinary skill in the art. Preferably, sequence analysis is performed by fragmentation of the peptides and computer-assisted interpretation of the fragment spectra using algorithms, e.g. MASCOT or SEQUEST. Both computer algorithms use protein and nucleotide sequence databases to perform cross-correlation analyses of experimental and theoretically generated tandem mass spectra. This allows automated high through-put sequence analysis.

Qualitative Peptide Analysis by MALDI Mass Spectrometry

For qualitative analysis of the whole peptide repertoire obtained upon elution, matrix-assisted laser desorption and ionization time-of-flight (MALDI-TOF) mass spectrometry may be carried out. Using settings that do not fragment the peptides, MALDI-TOF analysis provides a rough overview with regard to the complexity of the peptide mixture and the presence of dominant peptides.

Quantitative Peptide Analysis

To estimate the quantity of single peptides eluted from antigen presenting receptors, the run through of the micro-capillary column may be analyzed by a flow-through UV detector operated at a detection wave-length of 214 nm. For quantitation the peak areas of peptides to be analyzed are compared with peak areas of graded amounts of synthetic standard peptides.

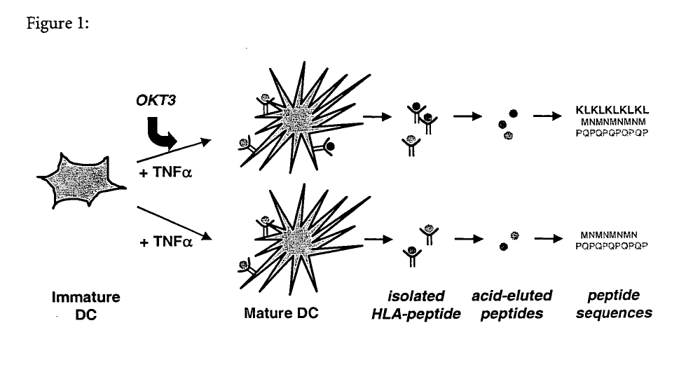

The present invention also is directed to identification of immunogenic peptides which have been loaded onto antigen presenting receptors of APCs in cell culture (in vitro approach, FIG. 1).

In a further embodiment the present invention relates to a method for identifying peptides involved in immunogenicity comprising the steps of
a) providing cells expressing antigen presenting receptors (APR) in a number providing 0.1 to 5 ug of said APR, preferably, in a number providing 0.2 to 3 ug of said APR,
b) contacting the cells from (a) with a source of immunogenic peptides, thus forming APR-immunogenic peptide complexes
c) isolating APR-immunogenic peptide complexes from the cells,
d) eluting the associated peptides from the APR.
e) identifying the immunogenic peptides; and
f) validating the identified immunogenic peptides as epitopes.

The APR expressing cells maybe MHC class II expressing cells (APCs). Preferably, APCs are dendritic cells, more preferably, the APCs are immature dendritic cells, most preferably, the APCs are immature dendritic cells generated from peripheral blood monocytes.

Dendritic cells may be generated from peripheral blood monocytes or from bone marrow-derived CD34+ stem cell-precursors. The peripheral blood mononuclear cells (PBMCs) may be isolated from blood samples by density gradient centrifugation. The monocytes may then be isolated from PBMCs by methods known in the art, e.g. by sorting with magnetic beads. The source of dendritic cells may be mammalian species, preferably humans. The monocytes may then be differentiated in cell culture to become immature dendritic cells. The differentiation state may be monitored by flow-cytometric analysis, e.g. using upregulation cell surface markers CD83, CD80, CD86, HLA-DR.

The amount of cells necessary to obtain e.g. 100 ng MHC class II molecules depends on the number of cells that do express MHC class II and on the expression rate of MHC class II molecules: e.g. 100 ng of MHC class II are equivalent to about 2×10⁵mature DCs or 5 to 10×10⁶ peripheral blood monocytes or about 5×10⁷ peripheral blood mononuclear cells which can be obtained from about 50 ml of blood. The APCs are then contacted with a source of therapeutic protein. The APCs, preferably the immature dendritic cells, are at the same time triggered to mature by methods known in the art, e.g. incubation with inflammatory cytokines, like TNF alpha or a mixture of TNF alpha, IL-6, IL-1 beta, PGE2.

The source of therapeutic protein offered to the APCs may be selected from the group comprising unformulated or formulated protein. Control APCs are treated equivalently except that they are not exposed to the therapeutic protein (cf. FIG. 1).

The APCs maybe contacted with the polypeptide or a fragment thereof which is taken up by the APCs by receptor-mediated uptake or by fluid phase uptake and internalized.

By eluting the peptides from the MHC molecules, a set of naturally processed peptides derived from the polypeptide or a fragment thereof is obtained. This polypeptide may be the therapeutic polypeptide of choice or an irrelevant polypeptide of intracellular (a self protein expressed in the APC in the absence of pulsed therapeutic polypeptide) or extracellular origin (a protein derived from the cell culture medium also present in the absence of pulsed therapeutic polypeptide).

The isolated immunogenic peptides may be identified by comparing the peptide identified from cells which have been contacted with a source of potential immunogen with those, which have been identified from cells which have not been contacted with that source (control).

Epitope Validation for MHC-Associated Peptides

The peptide sequences identified by the methods of the invention may be validated by one of several criteria, comprising MHC binding motif, MHC binding capacity and recognition by CD4+ T lymphocytes.

MHC binding motifs are common structural characteristics of peptides associated to a particular MHC molecule (allelic variant) which are necessary to form stable complexes with MHC molecules. In the case of MHC class II molecules, the peptide length varies from 12 to 18 amino acids and even longer peptides can bind since both ends of the peptide binding groove are open. Most HLA class II molecules accommodate up to 4 residues relevant for binding, denoted as “anchor residues”, at relative positions P1, P4, P6 and P9 contained in a nonameric core region. This core region, however, can have variable distance from the N-terminus of the peptide. In the majority of cases, 2-4 N-terminal residues precede the core region. Hence, the P1 anchor residues is located at positions 3, 4 or 5 in most HLA class II associated peptides. Peptides eluted from HLA-DR class II molecules share a big hydrophobic P1 anchor, represented by tyrosine, phenylalanine, tryptophane, methionine, leucine, isoleucine or valine.

The position and the exact type of anchor residues constitute the peptide binding motif which is known for most of the frequently occurring HLA-DR class II allelic products. A computer algorithm allowing motif validation in peptide sequences is “Tepitope”, available by www.vaccinome.com (by J. Hammer, Nutley, USA).

The MHC binding capacity of the peptides identified by the methods of the present invention may be tested by methods known in the art using, for example, isolated MHC class II molecules and synthetic peptides with amino acid sequences identical to those identified by the method of the invention (Kropshofer H et al., J. Exp. Med. 1992; 175, 1799-1803; Vogt A B et al., J. Immunol. 1994; 153, 1665-1673; Sloan V S et al., Nature 1995; 375, 802-806). Alternatively, a cellular binding assay using MHC class II expressing cell lines and biotinylated peptides can be used to verify the identified epitope (Arndt S O et al., EMBO J., 2000; 19, 1241-1251)

In both assays, the relative binding capacity of a peptide is measured by determining the concentration necessary to reduce binding of a labeled reporter peptide by 50% (IC50). Peptide binding with a reasonable affinity to the relevant HLA class II molecules attains IC50 values not exceeding 10-fold the IC50 of established reference peptides.

The same binding assays can also be used to test the ability of peptides to bind to alternative class II MHC molecules, i.e., class II MHC molecules other than those from which they were eluted using the method of the invention.

The capacity to prime CD4+ T cells represents the most critical epitope verification procedure. This procedure involves testing of peptides identified by the methods of the invention for their ability to activate CD4+ T cell populations. Peptides with amino acid sequences either identical to those identified by the methods of the invention or corresponding to a core sequence derived from a nested group of peptides identified by the methods of the invention are synthesized. The synthetic peptides are then tested for their ability to activate CD4+ in the context auf autologous dendritic cells, expressing the MHC class II molecule of interest.

CD4+ T cell responses can be measured by a variety of in vitro methods known in the art. For example, whole peripheral blood mononuclear cells (PBMC) can be cultured with and without a candidate synthetic peptide and their proliferative responses measured by, e.g., incorporation of [3H]-thymidine into their DNA. That the proliferating T cells are CD4+ T cells can be tested by either eliminating CD4+ T cells from the PBMC prior to assay or by adding inhibitory antibodies that bind to the CD4+ molecule on the T cells, thereby inhibiting proliferation of the latter. In both cases, the proliferative response will be inhibited only if CD4+ T cells are the proliferating cells. Alternatively, CD4+ T cells can be purified from PBMC and tested for proliferative responses to the peptides in the presence of APC expressing the appropriate MHC class II molecule. Such APCs can be B-lymphocytes, monocytes, macrophages, or dendritic cells, or whole PBMC. APCs can also be immortalized cell lines derived from B-lymphocytes, monocytes, macrophages, or dendritic cells. The APCs can endogenously express the MHC class II molecule of interest or they can express transfected polynucleotides encoding such molecules. In all cases the APCs can, prior to the assay, be rendered non-proliferative by treatment with, e.g., ionizing radiation or mitomycin-C.

As an alternative to measuring cell proliferation, cytokine production by the CD4+ T cells can be measured by procedures known to those in art. Cytokines include, without limitation, interleukin-2 (IL-2), interferon-gamma (IFN-gamma), interleukin-4 (IL-4), TNF-alpha, interleukin-6 (IL-6), interleukin-10 (IL-10), interleukin-12 (IL-12) or TGF-beta. Assays to measure them include, without limitation, ELISA, ELISPOT and bio-assays in which cells responsive to the relevant cytokine are tested for responsiveness (e.g., proliferation) in the presence of a test sample.

Applications

The methods of the present invention can be applied to identify peptides involved in the immunogenicity of any biopharmaceutical drug, especially those in which unacceptable potency loss is due to neutralizing anti-drug antibodies or where adverse or severe adverse events in clinical trials are thought to rely on immunogenicity.

The identified immunogenic peptides can further be used to de-risk the respective (therapeutic) polypeptides with regard to their immunogenicity. De-risking may be accomplished by exchange of one or more anchor residues critical for binding to MHC class II molecules, thereby creating mutated therapeutic polypeptides that have reduced or no immunogenicity potential. Alternatively, residues critical for recognition by the T cell receptor on CD4+ T cells can be exchanged.

Methods for exchanging anchor residues critical for binding to MHC class II molecules are well known in the art, i.e. replacement of the P1 anchor of a HLA-DR1-restricted T cell epitope by alanine, glycine, proline or a charged residue (cf. Kropshofer et al., EMBO J. 15, 6144-6154; 1996).

The methods of this invention can be used to reduce the number of epitopes that are being identified through in silico epitope prediction algorithms. Prediction codes tend to over-predict the number of epitopes contained in therapeutic polypeptides. The consequence of such an over-prediction is that de-risking of high numbers of predicted epitopes may lead to loss of bioactivity in those cases where certain sequence stretches confer both bioactivity and immunogenicity. As the present invention identifies naturally presented peptide epitopes, which have undergone competition for MHC binding sites and quality control by the peptide editor HLA-DM inside the APC, the methods presented here narrow down the number of potential epitopes to a reasonably small number. De-risking of a reduced number of epitopes will more likely retain the bioactivity of therapeutic polypeptides.

Having now generally described this invention, the same will become better understood by reference to the specific examples, which are included herein for purpose of illustration only and are not intended to be limiting unless otherwise specified, in connection with the following figures.

### EXAMPLES

The examples below are in connection with the figures described above and based on the methodology summarized in FIG. 1 and described in detail in the following. Commercially available reagents referred to in the examples were used according to manufacturer's instructions unless otherwise indicated.

### EXEMPLIFIED METHODOLOGY OF THE INVENTION

Cell Lines and Culture

The study was performed with human dendritic cells which were differentiated from monocytes, as described below. Monocytes were purified from human peripheral blood. All cells were cultured in RPMI 1640 medium (short: RPMI) supplemented with 1 mM Pyruvat, 2 mM Glutamine and 10% heat-inactivated fetal calf serum (Gibco BRL, Rockville, Md.).

Isolation of Peripheral Blood Mononuclear Cells (PBMCs)

Peripheral blood was obtained from the local blood bank as standard buffy coat preparations from healthy donors. Heparin (200 I.U./ml blood, Liquemine, Roche) was used to prevent clotting. Peripheral blood mononuclear cells (PBMCs) were isolated by centrifugation in LSM® (1.077-1.080 g/ml; ICN, Aurora, Ohio) at 800 g (room temperature) for 30 min. PBMCs were collected from the interphase and washed twice in RPMI containing 20 mM Hepes (500 g for 15 min, 300 g for 5 min). In order to remove erythrocytes, PBMCs were treated with ALT buffer (140 mM ammonium chloride, 20 mM Tris, pH 7.2) for 3 min at 37° C. PBMCs were washed twice with RPMI containing 20 mM Hepes (200 g for 5 min).

HLA-Typing of Peripheral Blood Monocytes

The HLA-DR genotype of PBMCs used for isolation of monocytes and differentiation of dendritic cells was determined by Roche Molecular Systems (Alameda, Calif., USA).

Generation of Dendritic Cells from Peripheral Blood Monocytes

Monocytes were isolated from PBMCs by positive sorting using anti-CD14 magnetic beads (Miltenyi Biotech, Auburn, Calif.) according to the manufacturer's protocol. Monocytes were cultured in RPMI supplemented with 1% non-essential amino acids (Gibco, BRL, Rockville, Md.), 50 ng/ml recombinant human granulocyte macrophage-colony stimulating factor (GM-CSF; S.A. 1.1×10⁷U/mg) (Leucomax; Novartis, Basel Switzerland) and 3 ng/ml recombinant human IL-4 (S.A. 2.9×10⁴ U/mg) (R&D Systems, Minneapolis, Minn.). Monocytes were seeded at 0.3×10⁶/ml in 6-well plates (Costar) for 5 days to obtain immature dendritic cells.

The quality of monocyte-derived immature dendritic cells was routinely monitored by flow-cytometric analysis conforming to the phenotype: CD1a (high), CD3 (neg.), CD14 (low), CD19 (neg.), CD56 (neg.), CD80 (low), CD83 (neg.), CD86 (low) and HLA-DR (high). In contrast, mature dendritic cells (cf. below) display the following phenotype: CD1a (low), CD80 (high), CD83 (high), CD86 (high) and HLA-DR (high). Monoclonal antibodies against CD1a, CD3, CD14, CD19, CD56, CD80, CD83, CD86 as well as the respective isotype controls were purchased from Pharmingen (San Diego, Calif.).

Exposure of Dendritic Cells to the Therapeutical Polypeptide

To facilitate the uptake of the pharmaceutical protein by dendritic cells, 6×10⁶ immature dendritic cells were exposed to 5-50 ug of the biopharmaceutical. At the same time, maturation of dendritic cells was induced by adding 10 ng/ml recombinant human tumor necrosis factor (TNFalpha; S.A. 1.1×10⁵ U/mg). As a control, 6×10⁶ dendritic cells were incubated with TNFalpha alone (FIG. 1)

After 24-48 hrs of co-culture, mature dendritic cells were harvested by centrifugation at 300 g for 10 min. Cells were washed with RPMI containing 10% FCS and transferred to an eppendorf tube. After centrifugation at 400 g for 3 min, the supernatant was completely removed and the cells were frozen at −70° C.

Generation of Anti-HLA Class II Beads

The anti-HLA-DR monoclonal antibody (mAb) L243 (ATCC, Manassas, Va.) was produced by culturing the respective mouse hybridoma cell line. mAb L243 was purified using ProteinA sepharose (Pharmacia, Uppsala, Sweden) and immobilized to CNBr-activated sepharose beads (Pharmacia) at a final concentration of 2.5 mg/ml, according to the manufacturer's protocol. L243 beads were stored in PBS containing 0.1% Zwittergent 3-12 (Calbiochem, La Jolla, Calif.).

Nano-Scale Purification of HLA-DR-Peptide Complexes

Pellets of frozen dendritic cells were resuspended in 10-fold volume of ice cold lysis buffer (1% Triton-X-100, 20 mM Tris, pH 7.8, 5 mM MgCl₂, containing protease inhibitors chyrnostatin, pepstatin, PMSF and leupeptin (Roche, Mannheim, Germany)) and lysed in a horizontal shaker at 1000 rpm, 4° C. for 1 h. The cell lysate was cleared from cell debris and nuclei by centrifugation at 2000 g, 4° C. for 10 min. The lysate was co-incubated with L243 beads (5-10 μl L243 beads per 100 μl cell lysate) in a horizontal shaker at 1000 rpm, 4° C. for 2 hrs. Immunoprecipitated HLA-DR-peptide complexes bound to L243 beads were sedimented by centrifugation at 2000 g, 4° C. for 5 min and washed three times with 300 μl 0.1% Zwittergent 3-12 (Calbiochem) in PBS.

The efficacy of depletion of HLA-DR-peptide complexes was monitored by analyzing the respective cell lysates before and after immunoprecipitation. In parallel, aliquots of the beads were analyzed by western blotting using the anti-HLA-DRα-specific mAb 1B5 (Adams, T. E. et al., Immunology 50 (1983) 613-624).

Elution of HLA-DR-Associated Peptides

HLA-DR-peptide complexes bound to L243 beads were resuspended in 400 μl H₂O (HPLC-grade; Merck, Darmstadt, Germany), transferred to an ultrafiltration tube, Ultrafree MC, 30 kD cut-off (Millipore, Bedford, Mass.) and washed 10 times with 400 μl H₂O (HPLC-grade) by centrifugation for 2-4 min at 14000 rpm at 4° C. For eluting the bound peptides, 50 μl 0.1% trifluoracetic acid (Fluka, Buchs, Switzerland) in H₂O (HPLC-grade) was added and incubation was performed for 30 min at 37° C. Eluted peptides were collected in a new eppendorf tube by centrifugation of the ultrafiltration tube at 14000 rpm for 3 min at RT and immediately lyophilized in a Speed-Vac® vacuum centrifuge.

Fractionation of Peptides by Nano-HPLC

Lyophilized peptides eluted from HLA-DR molecules were resolved in 0.05% trifluoroacetic acid, 5% acetonitrile (Merck, Darmstadt, Germany) in H₂O, (HPLC-grade) and separated on a 75 μm×15 cm C18 PepMap capillary (C18; 3 μm; 100 Å) (LC-Packings, Amsterdam, Netherlands) connected to a FAMOS® autosampler and an ULTIMATE® nano-flow HPLC (Dionex, Olten, Switzerland). The following non-linear gradient at a constant flow rate of 200 nl/min was used: 0-40 min 5-50% system B; 40-50 min 50-90% system B. System A was 0.05% trifluoroacetic, 5% acetonitrile/H₂O and system B was 0.04% trifluoroacetic, 80% acetonitrile/H₂O. The separation was monitored via dual UV absorption at 214 nm and 280 nm. Fractions (400 nl) were collected using the fraction collector PROBOTT (BAI, Weiterstadt, Germany) and spotted onto an AnchorChip 600/384 MALDI-MS target (Bruker, Bremen, Germany).

Sequence Analysis of Peptides by Ion Trap MS/MS Mass Spectrometry

To perform high-throughput sequencing of complex peptide mixtures, the MudPIT (multidimensional protein identification technology) was used (Washburn M P et al., Nat Biotechnol 19 (2001), 242-247) which is based on a liquid chromatographic fractionation followed by mass spectrometric sequencing.

To this end, the lyophilized peptides eluted from HLA molecules were resuspended in a buffer containing 5% (v/v) acetonitrile, 0.5% (v/v) acetic acid, 0.012% (v/v) heptafluoro butyric acid (HFBA) and 5% (v/v) formic acid. The sample was separated on a fused-silica microcapillary column (100 μm i.d.×365 μm) generated by a Model P-2000 laser puller (Sutter Instrument Co., Novato, Calif.). The microcolumn was packed with 3 μm/C18 reverse-phase material (C18-ACE 3 μm [ProntoSIL 120-3-C18 ACE-EPS, Leonberg, Germany]) followed by 3 cm of 5 μm cation exchange material (Partisphere SCX;Whatman, Clifton, N.J.).

A fully automated 8-step gradient separation on an Agilent 1100 series HPLC (Agilent Technologies, Waldbronn, Germany) was carried out, using the following buffers: 5% ACN/0.02% HFBA/0.5% acetic acid (buffer A), 80% ACN/0.02% HFBA/0.5% acetic acid (buffer B), 250 mM ammonium acetate/5% ACN/0.02% HFBA/0.5% acetic acid (buffer C), and 1.5 M ammonium acetate/5% ACN/0.02% HFBA/0.5% acetic acid (buffer D). The first step of 106 min consisted of a 100 min gradient from 0 to 80% buffer B and a 6 min hold at 80% buffer B. The next 6 steps (106 min each) are characterized by the following profile: 5 min of 100% buffer A, 2 min of x % buffer C, 5 min of 100% buffer A, a 3 min gradient from 0 to 10% buffer B, a 55 min gradient from 10 to 35% buffer B, a 20 min gradient from 35 to 50% buffer B, a 16 min gradient from 50 to 80% buffer B. The 2 min buffer C percentages (x) in steps 2-7 were as follows: 10, 20, 30, 40, 70, 90, and 100%. Step 8 consisted of the following profile: a 5 min 100% buffer A wash, a 20 min salt wash with 100% buffer D and a 100 min gradient from 0-80% buffer B.

The HPLC column was directly coupled to a Finnigan LCQ ion trap mass spectrometer (Finnigan, Bremen, Germany) equipped with a nano-LC electrospray ionization source. Mass spectrometry in the MS-MS mode was performed according to the manufacturer's protocol. The identification of peptides was done by the SEQUEST algorithm against the swiss.fasta database.

In silico Prediction of Potential Epitopes by TEPITOPE

Prediction of potential T cell epitopes was achieved by using the TEPITOPE algorithm. The following search criteria were applied: threshold (1-3% for best scoring and 4-6% for moderate scoring natural ligands), peptide length (15 amino acid residues) and promiscuity (predicted to bind to at least 6 out of 9 alleles). To determine the degree of promiscuity the following 9 alleles were chosen in agreement with their frequent occurrence in the Caucasian population: HLA-DRB1*0101, *0301, *0401, *0701, *0801, *1101, *1305, *1501 and DRB5*0101. Membrane-spanning domains and signal peptides were not included in the epitope search.

T Cell Activation Assay

The preparation of CD4⁺ T cells from fresh PBMCs was performed by negative selection using a CD4⁺ T cell isolation kit from Miltenyi Biotech (Auburn, Calif., USA). The T cell population was >75% pure and >95% viable as judged by Trypan blue staining (Sigma-Aldrich). T cells were resuspended at 2×10⁶ cells/ml in AIM V medium (Gibco BRL, Rockville, Md.). Dendritic cells (DCs) were differentiated from PBMCs as described and cultured in complete Macrophage-SFM medium (Gibco BRL, Rockville, Md.). On day 4, immature DCs were stimulated with 10 μg/ml LPS (Sigma-Aldrich). On day 6, matured DCs were washed and resuspended in AIM V medium at 2×10⁵ cells/ml. For the co-culture, 0.1 ml CD4⁺ T cells (2×10⁵) and 0.1 ml autologous DCs (2×10⁴), both in AIM V medium, were mixed in a round-bottomed 96-well format plate. OKT3 mAb and Inflexal V® were added to a final concentration of 20 μg/ml and 1 μg/ml, respectively. Synthetic peptides were added to a final concentration of 20 μM. Each antigen was tested in triplicate. On day 5 of the co-culture, 10 μM 5-bromo-2′-deoxyuridine (BrdU) (Roche, Basel, Switzerland) was added to each well. After 24 hrs incubation, cultures were harvested and processed according to the manufacturer's protocol. T cell proliferation of cultures without added antigen was used as reference with an average stimulation index (SI) set to 1.

For restimulation of T cells, immature DCs (2-3×10⁶/ml) were frozen at −70° C. in 50% AB serum (Sigma-Aldrich), 40% RPMI and 10% DMSO (Sigma-Aldrich). At the time point of restimulation, DCs were defrosted, washed and cultivated for 2 days in the presence of 10 μg/ml LPS. On day 5 (1^{st} restimulation) or day 10 (2nd restimulation) of the DC/T cell co-culture, 0.1 ml AIM V medium was withdrawn from each sample well prior to adding 0.1 ml of defrosted, mature DCs (2×10⁴) in AIM V to the co-culture together with fresh protein or peptide antigen. IL-2 (Pharmingen, San Diego, Calif.) was added in a final concentration of 100 U/ml.

### Example 1

OKT3 was the first therapeutic antibody. It has been approved by the FDA in 1986. It is a CD3-specific mouse IgG2a antibody and widely used in the clinic as an immunosuppressive drug in transplantation (L. Chatenaud, 2003), type 1 diabetes (E. Masteller & J. Bluestone, 2002) and psoriasis (T. Udset et al., 2002). Despite the profound OKT3-induced immunosuppression, the occurrence of an anti-OKT3 response to the xenogeneic protein was one of the main drawbacks in early clinical trials promoting rapid clearance and neutralization of OKT3 (G. Goldstein, 1987). It has been reported that the incidence of immunogenicity is roughly 85% in studies involving OKT3-treated individuals (C. Pendley et al., 2003).

The strategy outlined in FIG. 1 was used to identify peptide epitopes of OKT3, presented by dendritic cells displaying the HLA-DR genotype HLA-DRB1*0401/1302.

To identify HLA-DRB1*0401/1302-restricted OKT3 epitopes, dendritic cells, expressing the genotype HLA-DRB1*0401/1302 were differentiated from peripheral blood monocytes and cultured at a concentration of 0.5×10⁶ cells/ml. 5×10⁶ dendritic cells were exposed to the antibody OKT3 at a concentration of 20 μg/ml. At the same time, maturation of dendritic cells was induced by adding TNFα (10 ng/ml). As a control, the same amount of dendritic cells was cultured in the absence of OKT3, but in the presence of TNFα. After an incubation period of 24 hrs, both sets of dendritic cells were lysed in detergent TX-100 and HLA-DR molecules were precipitated by using the anti-HLA-DR mAb L243 immobilized to sepharose beads. HLA-DR associated peptides were eluted with 0.1% TFA and analyzed by 2D-LS/MS-MS.

Sequence analysis of HLA-DRB1*0401/1302-associated ligands revealed 3 OKT3-derived epitopes, represented by 7 peptide sequences derived from OKT3 (Table 1). Two of the epitopes were derived from the κ light chain, one epitope was located in the heavy chain. The three epitopes associated to the haplotypes DRB1*0401/1302 were found in at least 2 independent experiments.

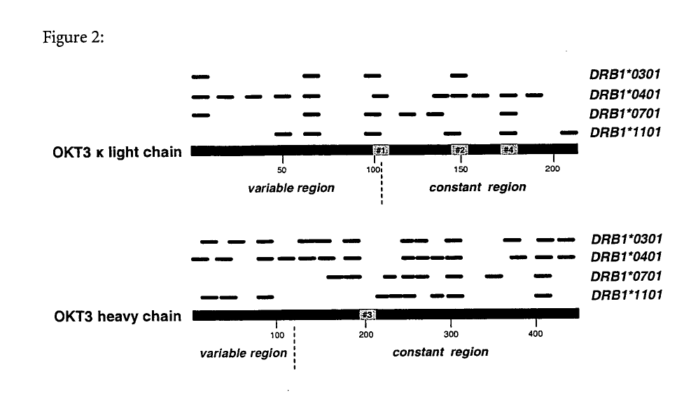

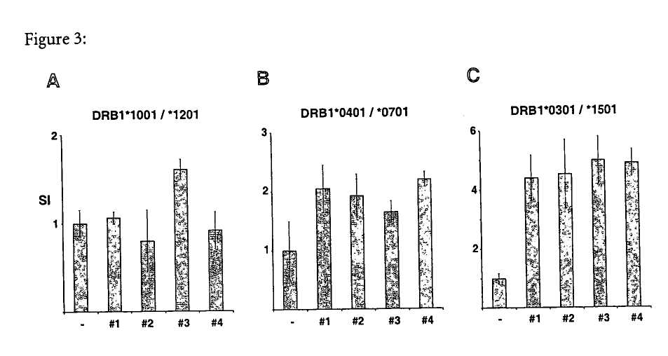

Epitope #1 was represented by a 15- and 16-mer peptide, the 15-mer being derived from the constant part of the light chain region 99-113 (Table 1). Epitope #1 contains the anchor motif of the DRB1*1302-associated co-dominant DRB3 allele DRB3*0301 (F. Verreck et al. 1996): L-103 as P1, N-106 as P4, A-108 as P6 and A-111 as P9 anchor. The same anchor residues may confer binding to DRB1*0401, as indicated by the TEPITOPE algorithm (FIG. 2). Epitope #1 was verified as a T cell epitope through its potency to induce proliferation of CD4+ T cells: Epitope #1 was stimulatory in context of dendritic cells that displayed the genotypes DRB1*0401/*0701 (FIG. 3B) and DRB1*0301/*1501 (FIG. 3C). However, it was incapable of activating T cells in context of the genotype DRB1*1001/*1201.

Epitope #2 was represented by 4 length variants: a 13-mer, a 14-mer, a 15-mer and a 16-mer peptide. This epitope was also derived from the constant region of the light chain subunit (Table 1). Epitope #2 was predicted by the TEPITOPE algorithm in the context of DRB1*0401 (FIG. 2) and contains the following anchor motif: W-147 as P1, D-150 as P4, S-152 as P6 anchor. In the T cell activation assay, epitope #2 stimulated proliferation of T cells in the context of the genotypes DRB1*0401/*0701 (FIG. 3B) and DRB1*0301/*1501 (FIG. 3C). However, it did not stimulate T cells in context of the genotype DRB1*1001/*1201. The homologous human sequence of epitope #2 was described in the context of the DRB1*0401 allele, extracted from an EBV-transformed B cell line (Friede et al., 1996).

Epitope #3 was represented by only one length variant: the 17-mer 194-210 was derived from the constant region of the OKT3 heavy chain (Table 1). Similar to epitope #1, epitope #3 contains the anchor motif of the DRB3 allele DRB3*0301: I-199 as P1, N-202 as P4, A-204 as P6 and A-207 as P9 anchor. Although the TEPITOPE algorithm did not predict epitope #3, neither for DRB1*0301, nor for *0401, *0701 or *1101 (FIG. 2), epitope #3 activated T cells in the context of all 3 DRB1 genotypes tested (FIG. 3). The same epitope has been described to be associated to the murine MHC class II molecule H2-A(s) (Rudensky et al., 1992).

When the TEPITOPE algorithm was employed to predict epitopes of the OKT3 kappa light chain in the context of the genotype DRB1*0401/1302, only predictions for DRB1*0401 could be made because the other alleles are not covered by the algorithm (FIG. 2). TEPITOPE predicted 11 epitopes in the kappa light chain, however, only two of them were among the naturally processed peptide epitopes, represented by epitopes #1 and #2 (FIG. 2). Likewise, TEPITOPE predicted 13 epitopes in the OKT3 heavy chain, however, none of them covered epitope #3 (FIG. 2).

### Example 2

The strategy outlined in FIG. 1 was also used to identify peptide epitopes of OKT3, presented by dendritic cells displaying the HLA-DR genotype HLA-DRB1*0701/1601.

To identify HLA-DRB1*0701/1601-restricted OKT3 epitopes, dendritic cells, expressing the genotype HLA-DRB1 *0701/1601 were differentiated from peripheral blood monocytes and cultured at a concentration of 0.5×10⁶ cells/ml. 5×10⁶ dendritic cells were exposed to the antibody OKT3 at a concentration of 20 μg/ml. At the same time, maturation of dendritic cells was induced by adding TNFα (10 ng/ml). As a control, the same amount of dendritic cells was cultured in the absence of OKT3, but in the presence of TNFα. After an incubation period of 24 hrs, both sets of dendritic cells were lysed in detergent TX-100 and HLA-DR molecules were precipitated by using the anti-HLA-DR mAb L243 immobilized to sepharose beads. HLA-DR associated peptides were eluted with 0.1% TFA and analyzed by 2D-LS/MS-MS.

Sequence analysis of HLA-DRB1*0701/1601-associated ligands revealed one OKT3-derived epitope, represented by 10 peptide sequences derived from OKT3 (Table 2). The epitope #4 was derived from the κ light chain and found in at least 2 independent experiments.

Epitope #4 was represented by a 10 length variants (15-22-mers) peptide, the 15-mer being derived from the constant part of the light chain region 168-182 (Table 2). Epitope #4 contains the anchor motif of the DRB1*0701 allele: Y-172 as P1, S-175 as P4, T-177 as P6 and L-180 as P9 anchor. The same anchor residues may confer binding to DRB1 *0401 and DRB1*1101, as indicated by the TEPITOPE algorithm (FIG. 2). Epitope #1 was verified as a T cell epitope through its potency to induce proliferation of CD4+ T cells: Epitope #4 was stimulatory in context of dendritic cells that displayed the genotypes DRB1*0401/*0701 (FIG. 3B) and DRB1*0301/*1501 (FIG. 3C). However, it was incapable of activating T cells in context of the genotype DRB1*1001/*1201.

Epitope #4 was recently described in the bovine system, extracted from blood mononuclear cells and presented by the bovine allele DRB3*2703 (Sharif et al., 2002).

When the TEPITOPE algorithm was employed to predict epitopes of the OKT3 kappa light chain in the context of the genotype DRB1*0701/*1601 only predictions for DRB1*0401 could be made because the DRB1*1601 allele is not covered by the algorithm (FIG. 2). TEPITOPE predicted 5 epitopes in the kappa light chain, however, only one of them was among the naturally processed peptide epitopes, represented by epitope #4 (FIG. 2). Likewise, TEPITOPE predicted 8 epitopes in the OKT3 heavy chain, however, none of them was supported by the analysis of naturally occurring peptides (FIG. 2).

### Example 3

The strategy outlined in FIG. 1 was used to identify peptide epitopes of OKT3, as recognized by T cells restricted by the HLA-DR genotypes HLA-DRB1*1101/1202.

To identify HLA-DRB1*1101/1202-restricted OKT3 epitopes, dendritic cells, expressing the genotype HLA-DRB1*1101/1202 were differentiated from peripheral blood monocytes and cultured at a concentration of 0.5×10⁶ cells/ml. 5×10⁶ dendritic cells were exposed to the antibody OKT3 at a concentration of 20 μg/ml. At the same time, maturation of dendritic cells was induced by adding TNFα (10 ng/ml). As a control, the same amount of dendritic cells was cultured in the absence of OKT3, but in the presence of TNFα. After an incubation period of 24 hrs, both sets of dendritic cells were lysed in detergent TX-100 and HLA-DR molecules were precipitated by using the anti-HLA-DR mAb L243 immobilized to sepharose beads. HLA-DR associated peptides were eluted with 0.1% TFA and analyzed by 2D-LS/MS-MS.

Sequence analysis of HLA-DRB1*1101/1202-associated ligands revealed 2 OKT3-derived epitopes, #1 and #3, represented by 6 peptide sequences derived from OKT3 (Table 3). One epitope was derived from the κ light chain the other epitope was located in the heavy chain. The two epitopes associated to the haplotypes DRB1*1101/1202 were found in at least 2 independent experiments.

Epitope #1 was represented by the same 15- and 16-mer peptide that has been described above in the context of the genotypes DRB1*0401/*1302 (cf. Tables 1 and 3). Epitope #1 contains the anchor motif of the DRB1*1101 allele: L-103 as P1, A-108 as P6 and A-111 as P9 anchor. Epitope #1 was verified as a T cell epitope through its potency to induce proliferation of CD4+ T cells: Epitope #1 was stimulatory in context of dendritic cells that displayed the genotypes DRB1*0401/*0701 (FIG. 3B) and DRB1*0301/*1501 (FIG. 3C). However, it was incapable of activating T cells in context of the genotype DRB1*1001/*1201.

Epitope #3, derived from the constant region of the OKT3 heavy chain (Tables 1,3), was represented by 4 length variants: the 14-mer 194-207, the 15-mer 194-208, the 17-mer 194-210 and the 18-mer 194-211 (Table 3). Although the TEPITOPE algorithm did not predict epitope #3, neither for DRB1*1101, nor for *1202 (FIG. 2), epitope #3 activated T cells in the context of all 3 DRB1 genotypes tested (FIG. 3).

When the TEPITOPE algorithm was employed to predict epitopes of the OKT3 kappa light chain in the context of the genotype DRB1*1101/1202, only predictions for DRB1*1101 could be made because the other alleles are not covered by the algorithm (FIG. 2). TEPITOPE predicted 5 epitopes in the kappa light chain, however, only one epitope was among the naturally processed peptide epitopes, represented by epitope #1 (FIG. 2). Likewise, TEPITOPE predicted 9 epitopes in the OKT3 heavy chain, however, none of them covered epitope #3 (FIG. 2).

### Example 4

The strategy outlined in FIG. 1 was also used to identify peptide epitopes of OKT3, presented by dendritic cells displaying the HLA-DR genotype HLA-DRB1*0301/0401.

To identify HLA-DRB1*0301/0401-restricted OKT3 epitopes, dendritic cells, expressing the genotype HLA-DRB1*0301/0401 were differentiated from peripheral blood monocytes and cultured at a concentration of 0.5×10⁶ cells/ml. 5×10⁶ dendritic cells were exposed to the antibody OKT3 at a concentration of 20 μg/ml. At the same time, maturation of dendritic cells was induced by adding TNFα (10 ng/ml). As a control, the same amount of dendritic cells was cultured in the absence of OKT3, but in the presence of TNFα. After an incubation period of 24 hrs, both sets of dendritic cells were lysed in detergent TX-100 and HLA-DR molecules were precipitated by using the anti-HLA-DR mAb L243 immobilized to sepharose beads. HLA-DR associated peptides were eluted with 0.1% TFA and analyzed by 2D-LS/MS-MS.

Sequence analysis of HLA-DRB1*0301/0401-associated ligands revealed one OKT3-derived epitope, represented by 1 peptide sequence derived from OKT3 (Table 2). The epitope #2 was derived from the κ light chain and found in at least 2 independent experiments.

Epitope #2 was represented by the 17-mer peptide 143-159 derived from the constant part of the light chain (Table 4). As described above (example 1), epitope #2 contains the anchor motif of the DRB1*0401 allele: W-147 as P1, D-150 as P4, S-152 as P6 anchor. The same anchor residues may confer binding to DRB1*0301, as indicated by the TEPITOPE algorithm (FIG. 2). Epitope #2 was verified as a T cell epitope through its potency to induce proliferation of CD4+ T cells: Epitope #2 was stimulatory in context of dendritic cells that displayed the genotypes DRB1*0401/*0701 (FIG. 3B) and DRB1*0301/*1501 (FIG. 3C). However, epitope #2 was incapable of activating T cells in context of the genotype DRB1*1001/*1201.

The TEPITOPE algorithm was employed to predict epitopes of the OKT3 kappa light chain in the context of the genotype DRB1*0301/*0401 (FIG. 2). TEPITOPE predicted 12 epitopes in the kappa light chain, however, only one of them was among the naturally processed peptide epitopes, represented by epitope #2 (FIG. 2). Likewise, TEPITOPE predicted 18 epitopes in the OKT3 heavy chain, however, none of them was supported by the analysis of naturally occurring peptides (FIG. 2).

### Example 5

Interferon-beta (IFN-β) is currently the first-line therapy for treatment of multiple sclerosis (Deisenhammer et al., 2000). Three different IFN-β formulations are currently marketed: Avonex, Rebif (both IFN-β-1a) and Betaseron (IFN-β-1b). Thereof Betaseron was the first one on the market, being approved by FDA in 1993 under accelerated approval regulations. In contrast to Avonex and Rebif, Betaseron is known to be exceptionally immunogenic. After treatment with Betaseron as much as 28-47% of patients produce anti-IFN-β neutralizing antibodies, while only 2-6% of Avonex-treated patients show neutralizing anti-drug antibodies (Deisenhammer et al., 2000; Bertolotto et al., 2004). In this context it is important to mention that Avonex and Rebif are expressed in Chinese hamster ovary cells as glycosylated proteins with the natural amino acid sequence, while Betaseron is expressed in E. coli in a non-glycosylated form with a Met-1 deletion and a Cys-17 to Ser point-mutation (Mark et al., 1984; Holliday and Benfield, 1997). So far it is unclear if these differences are responsible for the varying immunogenicity.

The strategy outlined in FIG. 1 was used to identify peptide epitopes of IFN-β-1b, presented by dendritic cells displaying the HLA-DR genotype HLA-DRB1*0101/0701.

To identify HLA-DRB1*0101/0701-restricted IFN-β-1b epitopes, dendritic cells, expressing the genotype HLA-DRB1*0101/0701 were differentiated from peripheral blood monocytes and cultured at a concentration of 0.5×10⁶ cells/ml. 5×10⁶ dendritic cells were exposed to IFN-β-1b at a concentration of 20 μg/ml. At the same time, maturation of dendritic cells was induced by adding lipopolysaccharide (LPS) at a concentration of 1 82 g/ml). As a control, the same amount of dendritic cells was cultured in the absence of IFN-β-1b, but in the presence of LPS. After an incubation period of 24 hrs, both sets of dendritic cells were lysed in detergent TX-100 and HLA-DR molecules were precipitated by using the anti-HLA-DR mAb L243 immobilized to sepharose beads. HLA-DR associated peptides were eluted with 0.1% TFA and analyzed by 2D-LS/MS-MS.

Sequence analysis of HLA-DRB1*0101/0701-associated ligands revealed one IFN-β-1b-derived epitope, #5, represented by 3 peptide sequences derived from IFN-β-1b (Table 5). The epitope #5 associated to the genotype DRB1*0101/0701 was also found in the context of the genotype DRB1*0101/1401 (cf. Table 8).

Epitope #5 was represented by a 13-mer, a 16-mer and a 17-mer peptide, the 13-mer being derived from the protein region 44-60 (Table 5). Epitope #5 contains the following anchor motif: F-49 as P1, E-52 as P4, A-54 as P6 and T-57 as P9 anchor. Consistently, in in vitro binding assays it has been shown that a 15-mer peptide containing epitope #5 has strong binding capabilities for the HLA allele DRB1*0101 (Tangri et al., 2005).

### Example 6

The strategy outlined in FIG. 1 was used to identify peptide epitopes of IFN-β-1b, presented by dendritic cells displaying the HLA-DR genotype HLA-DRB1*1101/1404.

To identify HLA-DRB1*1101/1404-restricted IFN-β-1b epitopes, dendritic cells, expressing the genotype HLA-DRB1*1101/1404 were differentiated from peripheral blood monocytes and cultured at a concentration of 0.5×10⁶ cells/ml. 5×10⁶ dendritic cells were exposed to IFN-β-1b at a concentration of 20 μg/ml. At the same time, maturation of dendritic cells was induced by adding lipopolysaccharide (LPS) at a concentration of 1 μg/ml). As a control, the same amount of dendritic cells was cultured in the absence of IFN-β-1b, but in the presence of LPS. After an incubation period of 24 hrs, both sets of dendritic cells were lysed in detergent TX-100 and HLA-DR molecules were precipitated by using the anti-HLA-DR mAb L243 immobilized to sepharose beads. HLA-DR associated peptides were eluted with 0.1% TFA and analyzed by 2D-LS/MS-MS.

Sequence analysis of HLA-DRB1*1101/1404-associated ligands revealed 2 IFN-β-1b-derived epitopes, #6 and #7, represented by 24 peptide sequences derived from IFN-β-1b (Table 6). Epitope #6 was also found in the context of the genotype DRB1*0801 (Table 7). Epitope #7 was also found in the context of genotype DRB1*0801 (Table 7), DRB1*0101/14 (Table 8) and DRB1*1303/1501 (Table 9).

Epitope #6 was represented by 22 length variants (11-19-mer), the 11-mer being derived from the protein region 89-99 (Table 6). Epitope #6 contains the following anchor motif: Y-91 as P1, I-94 as P4, H-96 as P6 and T-99 as P9 anchor. These anchor residues may confer binding to the HLA alleles DRB1*1101 and DRB1*0801, as predicted by the TEPITOPE algorithm. Although a 15-mer peptide containing the epitope #6 has been shown to bind to DRB1*0701 there was no evidence for T cell activation in this HLA context (Barbosa et al., 2005).

Epitope #7 was represented by a 13-mer and a 15-mer peptide, the 13-mer being from the protein region 149-161 (Table 6). Epitope #7 contains the following anchor motif: F-153 as P1, I-156 as P4, R-158 as P6 and G-161 as P9 anchor. A 15-mer peptide containing the epitope #7 has also been shown to be a promiscuous binder with very strong binding capabilities for the HLA alleles DRB1*0101, DRB1*1101 and DRB1*1501 (Tangri et al., 2005). Furthermore is has been described that a peptide pool containing the epitope #7 induces T cell activation in a DRB1*0701 background (Barbosa et al., 2005).

### Example 7

The strategy outlined in FIG. 1 was used to identify peptide epitopes of IFN-β-1b, presented by dendritic cells displaying the HLA-DR genotype HLA-DRB1*0801/0801.

To identify HLA-DRB1*0801/0801-restricted IFN-β-1b epitopes, dendritic cells, expressing the genotype HLA-DRB1*0801/0801 were differentiated from peripheral blood monocytes and cultured at a concentration of 0.5×10⁶ cells/ml. 5×10⁶ dendritic cells were exposed to IFN-β-1b at a concentration of 20 μg/ml. At the same time, maturation of dendritic cells was induced by adding lipopolysaccharide (LPS) at a concentration of 1 μg/ml). As a control, the same amount of dendritic cells was cultured in the absence of IFN-β-1b, but in the presence of LPS. After an incubation period of 24 hrs, both sets of dendritic cells were lysed in detergent TX-100 and HLA-DR molecules were precipitated by using the anti-HLA-DR mAb L243 immobilized to sepharose beads. HLA-DR associated peptides were eluted with 0.1% TFA and analyzed by 2D-LS/MS-MS.

Sequence analysis of HLA-DRB1*0801/0801-associated ligands revealed 2 IFN-β-1b-derived epitopes, represented by 22 peptide sequences derived from IFN-β-1b (Table 7). The two epitopes associated to the genotype DRB1*0801/0801 were found in at least 2 independent experiments.

Epitope #6 was represented by 17 length variants (11-18-mer), the 11-mer being derived from the protein region 89-99 (Table 6). As described above for DRB1*1101/1404 epitope #6 contains the following anchor motif: Y-91 as P1, I-94 as P4, H-96 as P6 and T-99 as P9 anchor. These anchor residues may confer binding to the HLA alleles DRB1* 1101 and DRB1*0801, as predicted by the TEPITOPE algorithm.

Epitope #7 was represented by the following 5 length variants: The 11-mer 151-161, the 13-mer 149-161, the 14-mer 149-162, the 14-mer 148-161 and the 15-mer 147-161 (Table 7). Epitope #7 contains the following anchor motif: F-153 as P1, I-156 as P4, R-158 as P6 and G-161 as P9 anchor.

### Example 8

The strategy outlined in FIG. 1 was used to identify peptide epitopes of IFN-β-1b, presented by dendritic cells displaying the HLA-DR genotype HLA-DRB1*0101/1401. To identify HLA-DRB1*0101/1401-restricted IFN-β-1b epitopes, dendritic cells, expressing the genotype HLA-DRB1*0101/1401 were differentiated from peripheral blood monocytes and cultured at a concentration of 0.5×10⁶ cells/ml. 5×10⁶ dendritic cells were exposed to IFN-β-1b at a concentration of 20 μg/ml. At the same time, maturation of dendritic cells was induced by adding lipopolysaccharide (LPS) at a concentration of 1 μg/ml). As a control, the same amount of dendritic cells was cultured in the absence of IFN-β-1b, but in the presence of LPS. After an incubation period of 24 hrs, both sets of dendritic cells were lysed in detergent TX-100 and HLA-DR molecules were precipitated by using the anti-HLA-DR mAb L243 immobilized to sepharose beads. HLA-DR associated peptides were eluted with 0.1% TFA and analyzed by 2D-LS/MS-MS.

Sequence analysis of HLA-DRB1*0101/1401-associated ligands revealed 2 IFN-β-1b-derived epitopes, represented by 9 peptide sequences derived from IFN-β-1b (Table 8). The two epitopes associated to the genotype DRB1*0101/1401 were found in at least 2 independent experiments.

Epitope #5 was represented by 7 length variants: The 15-mer 46-60, the 16-mer 45-60, the 17-mer 44-60, the 18-mer 43-60, the 19-mer 43-61, the 19-mer 42-60 and the 22-mer 39-60 (Table 8). Epitope #5 contains the following anchor motif: F-49 as P1, E-52 as P4, A-54 as P6 and T-57 as P9 anchor.

Epitope #7 was represented by a 13-mer and a 15-mer peptide, the 13-mer being from the protein region 149-161 (Table 8). Epitope #7 contains the following anchor motif: F-153 as P1, I-156 as P4, R-158 as P6 and G-161 as P9 anchor.

### Example 9

The strategy outlined in FIG. 1 was used to identify peptide epitopes of IFN-β-1b, presented by dendritic cells displaying the HLA-DR genotype HLA-DRB1*1303/1501.

To identify HLA-DRB1*1303/1501-restricted IFN-β-1b epitopes, dendritic cells, expressing the genotype HLA-DRB1*1303/1501 were differentiated from peripheral blood monocytes and cultured at a concentration of 0.5×10⁶ cells/ml. 5×10⁶ dendritic cells were exposed to IFN-β-1b at a concentration of 20 μg/ml. At the same time, maturation of dendritic cells was induced by adding lipopolysaccharide (LPS) at a concentration of 1 μg/ml). As a control, the same amount of dendritic cells was cultured in the absence of IFN-β-1b, but in the presence of LPS. After an incubation period of 24 hrs, both sets of dendritic cells were lysed in detergent TX-100 and HLA-DR molecules were precipitated by using the anti-HLA-DR mAb L243 immobilized to sepharose beads. HLA-DR associated peptides were eluted with 0.1% TFA and analyzed by 2D-LS/MS-MS.

Sequence analysis of HLA-DRB1*1303/1501-associated ligands revealed 1 IFN-β-1b-derived epitope, represented by three peptide sequences derived from IFN-β-1b (Table 9). The epitope associated to the genotype DRB1*1303/1501 was found in at least 2 independent experiments.

Epitope #7 was represented by the 13-mer 149-161, the 16-mer 148-161 and the 147-161 (Table 9). Epitope #7 contains the following anchor motif: F-153 as P1, I-156 as P4, R-158 as P6 and G-161 as P9 anchor.

TABLE 1

OKT-3 (Orthoclone) epitopes associated to the
genotype HLA-DRB1*0401/*1302.

| | | | | SEQ. |
|---|---|---|---|---|
| Epitope | | OKT-3 | OKT-3 | ID. |
| no. | Peptide sequence | subunit | region | NO |
| #1 | SGTKLEINRADTAPT | κ light | 99-113 | 3 |
| | | chain | | |
| | GSGTKLEINRADTAPT | | 98-113 | 4 |
| #2 | VKWKIDGSERQNG | κ light | 145-157 | 5 |
| | | chain | | |
| | NVKWKIDGSERQNG | | 144-157 | 6 |
| | INVKWKIDGSERQNG | | 143-157 | 7 |
| | INVKWKIDGSERQNGV | | 143-158 | 8 |
| #3 | WPSQSITCNVAHPASST | heavy | 194-210 | 9 |
| | | chain | | |

TABLE 2

OKT-3 (Orthoclone) epitopes associated to the
genotype HLA-DRB1*0701/*1601.

| Epi- | | | | SEQ. |
|---|---|---|---|---|
| tope | | OKT-3 | OKT-3 | ID. |
| no. | Peptide sequence | subunit | region | NO |
| #4 | KDSTYSMSSTLTLTK | κ light | 168-182 | 10 |
| | | chain | | |
| | KDSTYSMSSTLTLTKD | | 168-183 | 11 |
| | KDSTYSMSSTLTLTKDE | | 168-184 | 12 |
| | SKDSTYSMSSTLTLTKD | | 167-183 | 13 |
| | SKDSTYSMSSTLTLTKDE | | 167-184 | 14 |
| | DSKDSTYSMSSTLTLTKD | | 166-183 | 15 |
| | DSKDSTYSMSSTLTLTKDE | | 166-184 | 16 |
| | DQDSKDSTYSMSSTLTLTKD | | 164-183 | 17 |
| | DQDSKDSTYSMSSTLTLTKDE | | 164-184 | 18 |
| | TDQDSKDSTYSMSSTLTLTKDE | | 163-184 | 19 |

TABLE 3

OKT-3 (Orthoclone) epitopes associated to the
genotype HLA-DRB1*1101/*1202.

| | | | | SEQ. |
|---|---|---|---|---|
| Epitope | | OKT-3 | OKT-3 | ID. |
| no. | Peptide sequence | subunit | region | NO |
| #1 | SGTKLEINRADTAPT | κ light | 99-113 | 20 |
| | | chain | | |
| | GSGTKLEINRADTAPT | | 98-113 | 21 |
| #3 | WPSQSITCNVAHPA | heavy | 194-207 | 22 |
| | | chain | | |
| | WPSQSITCNVAHPAS | | 194-208 | 23 |
| | WPSQSITCNVAHPASST | | 194-210 | 24 |
| | WPSQSITCNVAHPASSTK | | 194-211 | 25 |

TABLE 4

OKT-3 (Orthoclone) epitopes associated to the
genotype HLA-DRB1*0301/*0401.

| | | | | SEQ. |
|---|---|---|---|---|
| Epitope | | OKT-3 | OKT-3 | ID. |
| no. | Peptide sequence | subunit | region | NO |
| #2 | INVKWKIDGSERQNGVL | κ light | 143-159 | 26 |
| | | chain | | |

TABLE 5

Interferon-β-1b epitopes associated to the
genotype HLA-DRB1*0101/*0701.

| Epitope | | | SEQ. ID. |
|---|---|---|---|
| no. | Peptide sequence | IFN-β-1b region | NO |
| #5 | QFQKEDAALTIYE | 48-60 | 27 |
| | QLQQFQKEDAALTIYE | 45-60 | 28 |
| | KQLQQFQKEDAALTIYE | 44-60 | 29 |

TABLE 6

Interferon-β-1b epitopes associated to the
genotype HLA-DRB1*1101/*1404.

| Epitope | | IFN-β-1b | SEQ. ID. |
|---|---|---|---|
| no. | Peptide sequence | region | NO |
| #6 | NVYHQINHLKT | 89-99 | 30 |
| | NVYHQINHLKTV | 89-100 | 31 |
| | NVYHQINHLKTVL | 89-101 | 32 |
| | NVYHQINHLKTVLE | 99-102 | 33 |
| | NVYHQINHLKTVLEE | 89-103 | 34 |
| | NVYHQINHLKTVLEEK | 89-104 | 35 |
| | ANVYHQINHLKT | 88-99 | 36 |
| | ANVYHQINHLKTV | 88-100 | 37 |
| | ANVYHQINHLKTVL | 88-101 | 38 |
| | ANVYHQINHLKTVLE | 88-102 | 39 |
| | ANVYHQINHLKTVLEE | 88-103 | 40 |
| | ANVYHQINHLKTVLEEK | 88-104 | 41 |
| | LANVYHQINHLKTV | 87-100 | 42 |
| | LANVYHQINHLKTVL | 87-101 | 43 |
| | LANVYHQINHLKTVLE | 87-102 | 44 |
| | LANVYHQINHLKTVLEE | 87-103 | 45 |
| | LLANVYHQINHLKTVL | 86-101 | 46 |
| | LLANVYHQINHLKTVLE | 86-102 | 47 |
| | LLANVYHQINHLKTVLEE | 86-103 | 48 |
| | LLANVYHQINHLKTVLEEK | 86-104 | 49 |
| | NLLANVYHQINHLKTVLE | 85-102 | 50 |
| | NLLANVYHQINHLKTVLEE | 85-103 | 51 |
| #7 | ILRNFYFINRLTG | 149-161 | 52 |
| | VEILRNFYFINRLTG | 147-161 | 53 |

TABLE 7

Interferon-β-1b epitopes associated to the
genotype HLA-DRB1*0801/*0801.

| Epitope | | IFN-β-1b | SEQ. ID. |
|---|---|---|---|
| no. | Peptide sequence | region | NO |
| #6 | NVYHQINHLKT | 89-99 | 54 |
| | NVYHQINHLKTV | 89-100 | 55 |
| | NVYHQINHLKTVL | 89-101 | 56 |
| | NVYHQINHLKTVLE | 99-102 | 57 |
| | NVYHQINHLKTVLEE | 89-103 | 58 |
| | ANVYHQINHLKT | 88-99 | 59 |
| | ANVYHQINHLKTV | 88-100 | 60 |
| | ANVYHQINHLKTVL | 88-101 | 61 |
| | ANVYHQINHLKTVLE | 88-102 | 62 |
| | LANVYHQINHLKT | 87-99 | 63 |
| | LANVYHQINHLKTV | 87-100 | 64 |
| | LANVYHQINHLKTVL | 87-101 | 65 |
| | LANVYHQINHLKTVLE | 87-102 | 66 |
| | LLANVYHQINHLKT | 86-99 | 67 |
| | LLANVYHQINHLKTVLE | 86-102 | 68 |
| | NLLANVYHQINHLKT | 85-99 | 69 |
| | NLLANVYHQINHLKTVLE | 85-102 | 70 |
| #7 | RNFYFINRLTG | 151-161 | 71 |
| | ILRNFYFINRLTG | 149-161 | 72 |
| | ILRNFYFINRLTGY | 149-162 | 73 |
| | EILRNFYFINRLTG | 148-161 | 74 |
| | VEILRNFYFINRLTG | 147-161 | 75 |

TABLE 8

Interferon-β-1b epitopes associated to the
genotype HLA-DRB1*0101/*1401.

| Epitope | | IFN-β-1b | SEQ. ID. |
|---|---|---|---|
| no. | Peptide sequence | region | NO |
| #5 | LQQFQKEDAALTIYE | 46-60 | 76 |
| | QLQQFQKEDAALTIYE | 45-60 | 77 |
| | KQLQQFQKEDAALTIYE | 44-60 | 78 |
| | IKQLQQFQKEDAALTIYE | 43-60 | 79 |
| | IKQLQQFQKEDAALTIYEM | 43-61 | 80 |
| | EIKQLQQFQKEDAALTIYE | 42-60 | 81 |
| | IPEEIKQLQQFQKEDAALTIYE | 39-60 | 82 |
| #7 | ILRNFYFINRLTG | 149-161 | 83 |
| | VEILRNFYFINRLTG | 147-161 | 84 |

TABLE 9

Interferon-β-1b epitopes associated to the
genotype HLA-DRB1*1303/*1501.

| Epitope | | IFN-β-1b | SEQ. ID. |
|---|---|---|---|
| no. | Peptide sequence | region | NO |
| #7 | ILRNFYFINRLTG | 149-161 | 85 |
| | EILRNFYFINRLTG | 148-161 | 86 |
| | VEILRNFYFINRLTG | 147-161 | 87 |

1. A method for identifying peptides involved in immunogenicity comprising:
a) providing cells expressing antigen presenting receptors (APR) in a number providing 0.1 to 5 ug APR molecules,
b) contacting the cells from (a) with a source of immunogenic peptides,
c) isolating APR molecule-immunogenic peptide complexes from the cells,
d) eluting the associated immunogenic peptides from the APR molecules; and
e) identifying the isolated immunogenic peptides.

2. The method according to claim 1, wherein the APR expressing cells are MHC II expressing cells.

3. The method according to claim 2, wherein the MHC II expressing cells are dendritic cells.

4. The method according to claim 1, wherein the source of immunogenic peptides is selected from the group consisting of cytokines, chemokines, growth factors, antibodies, enzymes, structural proteins, hormones and fragment(s) thereof.

5. The method according to claim 2, wherein the complexes of MHC II molecules with immunogenic peptides are isolated from the cells, said isolation comprising: a) solubilization of the cells with a detergent, and b) sequestration of the complexes of antigen presenting receptors with immunogenic peptides by immunoprecipitation or immunoaffinity chromatography.

6. The method according to claim 1, wherein the isolated immunogenic pep tides are identified by comparing the peptide identified from cells which have been contacted with a source of potential immunogen with those, which have been identified from cells which have not been contacted with that source.

7. The method according to claim 1, wherein the immunogenic peptides are naturally-processed immunogenic peptides.

8. A method for decreasing the immunogenicity of a polypeptide comprising
a) identifying the immunogenic peptides of the polypeptide according to the method of claim 1; and
b) modifying the corresponding epitopes of the polypeptide so that the binding to antigen presenting receptor is reduced or abolished.

9. The method of claim 1, further comprising validating the identified isolated immunogenic peptides as epitopes.
